(19) United States
(12) Patent Application Publication
(10) Pub. No.: US 2009/0177145 A1
(43) Pub. Date: Jul. 9, 2009

(54) MEDICAL DEVICE AND METHOD FOR MONITORING HEMATOCRIT AND SVO2

(76) Inventors: Malin Ohlander, Stockholm (SE); Kjell Noren, Solna (SE); Johan Eckerdal, Knivsta (SE); Mika Hietanen, Handen (SE); Kenth Nilsson, Akersberga (SE); Urban Lonn, Uppsala (SE)

Correspondence Address: SCHIFF HARDIN, LLP;PATENT DEPARTMENT, 6600 SEARS TOWER, CHICAGO, IL, 60606-6473, US

(21) Appl. No.: 12/373,356

(22) PCT Filed: Feb. 22, 2007
(86) PCT No.: PCT/SE2007/000165

§ 371 Date: Jan. 12, 2009

Related U.S. Application Data
(63) Continuation of application No. 11/363,922, filed on Feb. 28, 2006.

(30) Foreign Application Priority Data
Dec. 21, 2006 (EP) 06026522.0

(51) Int. Cl. A61M 37/00; G06Q 50/00; A61B 5/1459; A61B 5/02

(52) U.S. Cl. 604/66; 705/2; 600/327; 600/301

(57) ABSTRACT

A method, an implantable medical device, and a computer-readable medium encoded with programming instructions allow monitoring of a hematocrit value and an SvO2 level of a patient, making use of at least one medical lead connected to an implantable medical device that carries an optical sensor module that measures at least one hematocrit value and at least one SvO2 value using at least first, second and third light radiation wavelengths, by determining a present hematocrit value from at least one of the measured hematocrit values and determining a present SvO2 value from at least one of the measured SvO2 values, and determining a patient status by evaluating the present hematocrit value and the present SvO2 value, to allow a change in the patient status to be identified.

### BACKGROUND OF THE INVENTION

1. Field of the Invention

The present invention generally relates to cardiac pacing systems and, in particular, to a method and an implantable medical device for monitoring physiological parameters such as hematocrit and SvO2 levels of a patient to determine a patient status.

2. Description of the Prior Art

Physiological parameters such as hematocrit and SvO2 are common parameters used by physicians to diagnose and monitor patients. The hematocrit indicates the proportion of cells and fluids in the blood. The hematocrit is, in practice, the percent of whole blood that is composed of red blood cells (Erythrocytes). In men 39-55% of the blood volume is made up of red blood cells and in women 36-48%. A low hematocrit value may be the result from either an increased plasma volume (hemodilution) or from a reduced red blood cell volume (true anaemia). In patients suffering from CHF, low hematocrit values has been found to be of a frequent occurrence and is, also, associated with a poor prognosis, see, for example, “Hemodilution is Common in Patients with Advanced Heart Failure”, Androne et al., Circulation. 2003; 107:226-229.

In “The Cardio Renal Anaemia (CRA) Syndrome: Congestive Heart Failure, Chronic Kidney Insufficiency, and Anaemia”, Silverberg et al., Dialysis Times, News & Views from RPI, Volume 10, No. 1, it is shown that the presence of anaemia is associated with a more severe degree of CHF and with an increased mortality in CHF. In addition, it is shown that there exists a strong correlation between the severity of anaemia and hospitalization and/or length of stay. The anaemia caused by the CHF is not merely hemodilutional, but also due to a reduction in red cell volume, which, in turn, may be due to primarily two factors: the renal damage caused by the CHF causes a reduced production of EPO (erythropoietin) in the kidneys and CHF itself may cause anaemia. Animal studies have confirmed that anaemia is common in CHF.

In inflammatory bowel disease, anaemia is an indicator of disease severity and in HIV/AIDS patients anaemia has a serious impact on the quality of life of the patients and is also strongly associated with disease progression and in an increased risk of death.

SvO2 is a measure of a relation between oxygen delivery and oxygen consumption. SvO2 varies directly with cardiac output and SaO2 and inversely with VO2 (oxygen consumption). The normal SvO2 is about 75%, which indicates that under normal conditions, tissues extract 25% of the oxygen delivered. An increase in VO2 or a decrease in arterial oxygen content (SaO2×Hb) is compensated by increasing CO or tissue oxygen extraction. When the SvO2 is less than 30%, tissue oxygen balance is compromised and anaerobic metabolism ensues. A normal SvO2 does not ensure a normal metabolic state but suggests that oxygen kinetics are either normal or compensated. SvO2 is thus a global parameter indicating how well oxygenated the body is.

Hence, it would be a great benefit if a trend over the changes in hematocrit and SvO2 could be determined, for example, for a physician handling a patient suffering from a condition such as congestive heart failure (CHF), as well as for patients suffering from other diseases such as chronic kidney disease. Hematocrit and SvO2 can thus also be used to guide drug titration, monitor progression/regression of a disease and alert for deterioration of the patient.

Drug titration is an area where the treatment of the patients can be improved significantly. A number of outside factors such as the amount of exercise, food habits (consumption of coffee, salt alcohol etc) will change the amount of drugs required on a day to day basis. Currently the patient usually takes one dose of medication regardless these external factors, leading to an over consumption/under consumption of the drug. An over consumption is unbeneficial since the drugs often come with bi-effects and an under consumption would not lead to an effective therapy. Optimizing the drug consumption would therefore be very beneficial to the patient.

Monitoring the long term progression and regression of a disease is of essence for the physician to make therapeutic decisions for the patients. Also, having parameters such as hematocrit and SvO2 monitored continuously (and not just measurements taken during visits to the physician) would provide a better and more complete picture of the disease progression.

Another area of patient care that would benefit from monitoring a trend over SvO2 and hematocrit is alerting and avoiding acute de-compensation of the patient. Avoiding hospitalization would be very beneficial, if not life saving, to the patient but would also reduce costs for the society.

CHF patients are prone to have co-morbidities and the same or similar symptoms can thus be caused by many different pathophysiological factors, some are life threatening and require immediate attention and others are less damaging to the patient and may only require a change in medication or dosage of medication. Accordingly, there would be an advantage if further physiological parameters including body temperature, heart rate, activity and/or minute volume could be monitored to obtain an increased degree of sensitivity/specificity. Thereby, it would be possible to detect truly acute episodes from episodes that does not require immediate medical attention since a more global picture of the patient's health is obtained. For example, the limited cardiac output of a heart failure patient might alter body temperatures and the dynamics of response to altered thermal conditions and exercise. A specific example is the known tendency for body core temperature to fall when heart failure patients exercise.

Consequently, there is a need within the art of systems for monitoring hematocrit and SvO2 to determine a patient status in order to provide an improved patient care. It would also be an advantage if physiological parameters including body temperature, heart rate, activity and/or minute volume being indicative of the health status of a patient could be monitored and used to determine a patient status in order to provide a further improved patient care.

In light of this, a number of solutions in which different physiological parameters are monitored and used to determine a status or condition of a patient have been presented. In United States Patent Application Publication No. 2006/0149145 an apparatus including implantable optical sensors for measuring hematocrit and SvO2 is disclosed. The sensor is connected to an extracorporeal analyzing and monitoring device enabling a monitoring of blood characteristics over a long term to determine, for example, a condition of a patient.

Further, EP 1 107 158 discloses a system and method for determining a reference base line of patient status for an individual patient for use in an automated collection and analysis patient care system. A set of measures including SvO2 and hematocrit is collected from an implanted medical device and stored in an external database, which is organized to store one or more patient care records. The collected measures are processed into a set of reference measures, wherein each reference measure is representative of at least one of measured or derived patient information. The reference measures set is stored into the patient care record indicating an initial patient status.

In United States Patent Application Publication No. 2005/0153885 methods are disclosed for treating a patient for a condition caused by an abnormality in the autonomic nervous system by modulating the autonomic nervous system with at least one aldosterone antagonist. SvO2 or hematocrit is measured by means of implantable mechanical or electrical sensors in order to collect data for determining the modulation. The treatment can be performed by means of an implanted drug pump.

Accordingly, despite numerous solutions for sensing SvO2 and/or hematocrit in monitoring and diagnosing purposes, there has not hitherto been presented an overall solution taking all the above mentioned aspects of patient care into consideration and, thus, there is still a need within the art to be able to automatically collect SvO2 and hematocrit values of a patient and to determine a patient status using the collected values in monitoring and therapeutic purposes over a long term period.

### SUMMARY OF THE INVENTION

An object of the present invention is to provide an improved method and an implantable medical device for automatically collecting SvO2 and hematocrit values of a patient and for determining a patient status for monitoring and/or therapeutic purposes over a long term period.

Another object of the present invention is to provide an improved method and an implantable medical device for automatically collecting SvO2 and hematocrit values of a patient and determining a reliable and overall patient status for monitoring and/or therapeutic purposes.

A further object of the present invention is to provide an improved method and an implantable medical device that are capable of, in an accurate and reliable way, measuring the hematocrit and SvO2 of a patient in vivo using an optical sensor to detect or monitor a change of a condition of a patient, such as CHF, cancer, chronic kidney disease, diabetes, rheumatoid arthritis, inflammatory bowel disease and HIV/AIDS.

Still another object of the present invention is to provide an improved method and medical device that are capable of measuring the hematocrit and SvO2 on a substantially continuous basis, as well as during different time points of the day, for example, during the night.

Yet another object of the present invention is to provide a method and medical device that are capable of providing an improved patient care.

According to an aspect of the present invention, an implantable medical device for monitoring a hematocrit level and a SvO2 level of a patient connectable to at least one medical lead includes an optical sensor module adapted to measure at least one hematocrit value and at least one SvO2 value by means of at least a first, a second, and a third light radiation wavelength. The implantable medical device has a blood constituent determining device that obtains measured hematocrit values and SvO2 values, to determine a present hematocrit level by means of the at least one hematocrit value and to determine a present SvO2 level by means of the at least one SvO2 value, and a patient status determining device that determines a patient status based on an evaluation of the present hematocrit level and the present SvO2 level, wherein a change of a condition of the patient can be derived.

According to a second aspect of the present invention, a method for monitoring a hematocrit level and a SvO2 level of a patient is implemented in an implantable medical device connectable to at least one medical lead including an optical sensor module adapted to measure at least one hematocrit value and at least one SvO2 value by means of at least a first, a second, and a third light radiation wavelength. The method includes the steps of: measuring hematocrit values and SvO2 values; determining a present hematocrit level by means of the at least one hematocrit value and to determine a present SvO2 level by means of the at least one SvO2 value; and determining a patient status based on an evaluation of the present hematocrit level and the present SvO2 level, wherein a change of a condition of the patient can be derived.

According to a third aspect of the present invention, a computer-readable medium encoded with programming instructions is directly loadable into an internal memory of an implantable medical device, the programming instructions causing the implantable medical device to perform steps in accordance with the aforementioned method.

The basis of the present invention is to automatically monitor physiological parameters that provide a global picture of a patients health and status. SvO2 and hematocrit and the development of these parameters over time have been found to provide valuable information, for example, for a physician handling a patient suffering from a condition such as congestive heart failure (CHF), as well as for patients suffering from other diseases such as chronic kidney disease, and in use for guidance of drug titration, in monitoring of progression/regression of a disease, and in alerting for a deterioration of the patient. Monitoring the long term progression and regression of a disease is of essence for the physician to make therapeutic decisions for the patients. By continuously and automatically monitoring hematocrit and SvO2 (and not just measurements taken during visits to the physician) provides an improved and more complete picture of the disease progression. Another area of patient care that benefits from monitoring a trend over SvO2 and hematocrit is alerting and avoiding acute decompensation of the patient. Avoiding hospitalization is very beneficial, if not life saving, to the patient and may also reduce costs for the society. Hence, the present invention provides for an improved patient comfort taking a large number of different aspects of patient care into account.

According to an embodiment of the present invention, the blood constituents determining device is adapted to obtain measured hematocrit values and SvO2 values continuously or at predetermined intervals, wherein at least one sequence over time of hematocrit levels and SvO2 levels, respectively, can be determined.

In a further embodiment of the present invention, the implantable medical device further includes a therapy determining device that obtains a target range for the hematocrit level and the SvO2 level, respectively, and to compare the obtained target ranges with the present hematocrit level and the present SvO2 level, respectively, to determine a therapy for the patient. In one certain embodiment, the therapy determining device is adapted to determine a dosage of a drug, which may be performed on a continuous basis. Thereby, it is possible to optimize the drug dosage over time taking variations in the hematocrit level and the SvO2 level into consideration. A number of outside factors such as the amount of exercise, food habits (consumption of coffee, salt alcohol etc) will change the amount of drugs required on a day to day basis. Today, the patient usually takes one dose of medication regardless these external factors, leading to an over consumption/under consumption of the drug. An over consumption is unbeneficial since the drugs often come with bi-effects and an under consumption would not lead to an effective therapy. Optimizing the drug consumption would therefore be very beneficial to the patient. The determined dosage may be communicated to the patient via a communication unit of the implantable medical device and an external device such as a portable user equipment, e.g. a mobile phone, or a stationary home monitoring unit such as a programmer. Accordingly, the patient care can be further improved by dynamically determining a drug dosage over time.

In accordance with a further embodiment of the present invention, the therapy determining device is connected to a drug delivering device and is adapted to control the drug delivering device so as to deliver a drug to the patient based on the determined dosage and/or patient status. Thereby, the drug delivery can be adjusted automatically and continuously in response of changing physiological conditions of the patient such that an optimal drug dosage can be delivered despite outside factors such as the amount of exercise, food habits (consumption of coffee, salt alcohol etc) out of control for a physician determining the drug dosage at prescription of the medication. The physician can also be updated continuously and automatically with the current dosage via a monitoring device, e.g. a PC, connected to a communication network with which the implantable medical device is able to communicate with via an external device such as a user equipment (e.g. a mobile phone) or a home monitoring unit (e.g. a programmer). In addition, the patient may be updated continuously and automatically with the current dosage by means of the user equipment or the home monitoring unit.

According to embodiments of the present invention, the implantable medical device has further sensors that sense or measure other physiological parameters such as a body temperature sensor adapted to sense a body temperature of the patient. Thereby, the device is capable of obtaining body temperature values continuously or at predetermined intervals, wherein at least one sequence over time of body temperature values can be determined. The sensed body temperature values may be used in the evaluation of the present hematocrit level and the present SvO2 level to determine a patient status, wherein a change of a condition of the patient can be derived. Furthermore, the therapy or dosage of a drug or a progression/regression of a disease may also be determined by taking the body temperature, as well as hematocrit and SvO2 into account. Thereby, it is possible to provide a more thorough picture of the patient's health and a drug dosage can be determined with a higher degree of accuracy and reliability.

In yet another embodiment of the present invention, the implantable medical device has an activity sensor that senses an activity level of the patient. The device is accordingly capable of obtaining sensed activity levels continuously or at predetermined intervals, wherein at least one sequence over time of an activity level can be determined. The sensed activity levels can be used in the evaluation of the present hematocrit level and the present SvO2 level to determine a patient status, wherein a change of a condition of the patient can be derived. Moreover, the obtained activity levels can also be used, in addition to the hematocrit and SvO2 to determine a therapy, a dosage of a drug, or a progression/regression of a disease. Thereby, it is possible to provide a more thorough picture of the patient's health and a drug dosage can be determined with a higher degree of accuracy and reliability.

In another embodiment of the present invention, the implantable medical device comprises an impedance measuring circuit measures a transthoracic impedance, the impedance measuring circuit being connected to electrodes of the at least one medical lead and/or to a housing of the implantable medical device. The impedance measuring circuit, during impedance measurement sessions, generates electrical signals to be applied between at least a first electrode and at least a second electrode and to measure the impedance in the tissue between the at least first electrode and the at least second electrode to the applied electrical signals. The patient status determining device obtains sensed impedance values continuously or at predetermined intervals, wherein at least one sequence over time of impedance values can be determined, and to determine at least one sequence of a minute ventilation of the patient using the impedance values. The minute ventilation can be used to determine a patient status together with the hematocrit and SvO2, wherein a change of a condition of the patient can be derived. Further, the minute ventilation can also be used, in addition to the hematocrit and SvO2, to determine a therapy, a dosage of a drug, or a progression/regression of a disease. Thereby, it is possible to provide a more thorough picture of the patient's health and a drug dosage can be determined with a higher degree of accuracy and reliability.

In yet another embodiment of the present invention, a heart rate sensor is included in the implantable medical device adapted to sense a heart rate of the patient. The patient status determining device is adapted to obtain sensed heart rate level values continuously or at predetermined intervals, wherein a sequence over time of heart rate level values can be determined. The sensed heart rate can be used in the evaluation of hematocrit and SvO2, to determine a patient status, wherein a change of a condition of the patient can be derived. In addition, the heart rate can also be used, in addition to the hematocrit and SvO2, to determine a therapy, a dosage of a drug, or a progression/regression of a disease. For example, if it is found that SvO2 is within a predetermined range defining normal values for a particular patient, that hematocrit decreases and the heart rate increases, it is an indication of anaemia. Hence, it is possible to provide a more thorough picture of the patient's health and a drug dosage can be determined with a higher degree of accuracy and reliability.

In further embodiments of the present invention, one of, some of, or all of the parameters heart rate, patient posture, body temperature, activity, minute ventilation is (are) used together with the hematocrit and SvO2 to determine a patient status, a therapy, a dosage of a drug, or a progression/regression of a disease. For example, if it is found that SvO2 and hematocrit is within predetermined ranges, respectively, defining normal values for a particular patient, that body temperature increases and the heart rate increases, it is an indication of that the patient has an infection. Hence, it is possible to provide an even more thorough picture of the patient's health and, for example, a progression/regression of a condition or disease or a drug dosage can be determined with a higher degree of accuracy and reliability. Many heart failure patients have comorbities and the same symptom can originate for different disorders in the patient (dyspnea for instance can be a sign of volume overload in the lung but can also be a sign of poor oxygenation of the patient due to low hematocrit). Trends over several physiological parameters including heart rate, patient posture, body temperature, activity, minute ventilation, hematocrit and SvO2 will provide the physician with an efficient an accurate tool to establish the cause of a deterioration and grade of the severity of the problem.

Furthermore, different combinations of parameters can be used to provide indications of different conditions and a set of criteria may be defined for that purpose. Each criterion may give rise to an alert signal and there may hence be a number of different signals each signalling, for example, a crossing of certain parameter limit. The patient status determining device may send such an alert signal to the user equipment and/or the home monitoring unit informing the patient that he or she should see his/her physician.

Another use of a patient status determining device is in the common situation when a patient is under a home medical care program. An example is an elderly patient that lives at home and has a weekly visit by a nurse. The nurse will check the patient status and distribute the daily dosage of drugs for the week to come. It would be very beneficial to read out the trended data collected during the last week using a monitoring device. Changes in the patient status can then be alerted. Consultations with a physician may be initiated to change the dosage of drugs. Infections that need antibiotic treatment can be alerted. An example is urinary tract infections which untreated may lead to an infection of the kidneys (pyelonephritis). Acute pyelonephritis can be a severe conditions with a high mortality and in people who are immunosuppressed, for example, people suffering from cancer of AIDS.

Moreover, the therapy determining device and/or the patient status determining device may send an information message and/or alert signal, e.g. that a monitored parameter or a combination of monitored parameters has exceeded or fallen below the predetermined limits, respectively, to the patient and/or the physician. If the physician receives an alert signal together with collected data of the parameters, the physician/nurse may rate the level of acuteness of the deterioration and be guided whether the patient have to visit the hospital or care institution at once or within the next few weeks, or only be prescribed a new medication. It may also be established in an early phase which physician branch (nephrologist, cardiologist, pulmonologist, internal medicine) the patient should see if a hospital visit is required. It would also be beneficial in an in-clinic scenario where the information could eliminate certain pathophysiological factors and thus eliminate tests thereby reducing costs and provide the physician with the relevant information. Also, in a regular follow-up it would provide insight to patients general health and help guide the physician of the overall therapy. In a remote follow-up scenario it would provide the physician with physiological/hemodynamic information to provide better quality of the follow-up.

A patient may also be notified on a situation where a monitored parameter or a combination of monitored parameters assumes abnormal values. This may be done, for example, by causing a vibration unit of the implantable medical device to vibrate thereby informing the patient of the event, or sending a signal to an external device such as a user equipment, e.g. a mobile phone, a personal digital assistant or a pager, or a home monitoring unit. The message may be in form of a text message informing the patient of the event or a signal causing a lamp to start twinkle. Thus, the patient may be alerted that he or she should contact his or hers physician.

As will be understood by those skilled in the art, steps of the methods of the present invention, as well as preferred embodiment thereof, are suitable to realize as a computer program or a computer readable medium.

The features that characterize the invention, both as to organization and to method of operation, together with further objects and advantages thereof, will be better understood from the following description used in conjunction with the accompanying drawings. It is to be expressly understood that the drawings is for the purpose of illustration and description and is not intended as a definition of the limits of the invention. These and other objects attained, and advantages offered, by the present invention will become more fully apparent as the description that now follows is read in conjunction with the accompanying drawings.

### BRIEF DESCRIPTION OF THE DRAWINGS

FIG. 1 schematically shows an embodiment of a pacemaker system in which an implantable medical device in accordance with the present invention may be implemented.

FIG. 2 schematically shows an embodiment of an implantable medical device in accordance with the present invention.

FIG. 3 schematically shows a medical system in accordance with an embodiment of the present invention including the implantable medical device shown in FIG. 2.

FIG. 4 shows an optical sensor module which may be implemented in a medical lead connectable to the implantable medical device shown in FIG. 2.

FIG. 5a illustrates the principles of the oxygen saturation measurements using the sensor module of FIG. 4.

FIG. 5b illustrates the principles of the hematocrit measurements using the sensor module of FIG. 4.

FIG. 5c illustrates the principles of the hematocrit measurements using the sensor module of FIG. 4.

FIG. 5d illustrates the principles of the calibration of the sensor module of FIG. 4.

FIG. 6 is a high-level description of the method according to the present invention.

FIG. 7 is a high-level description of an exemplary embodiment of the method according to the present invention.

FIG. 8 is a high-level description of another embodiment of the method according to the present invention.

### DESCRIPTION OF THE PREFERRED EMBODIMENTS

In the following, the present invention will be discussed in the context of medical systems comprising at least an implantable medical device such as a pacemaker or an ICD, and connectable to medical leads such as an atrial lead and a ventricular lead.

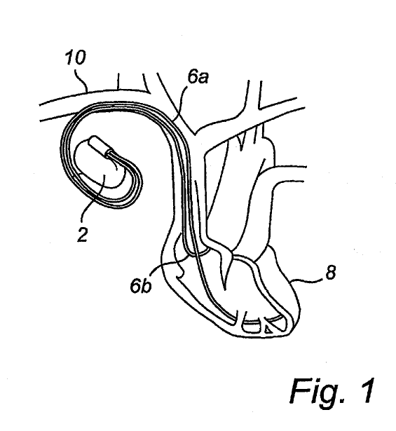

With reference to FIG. 1, there is shown a schematic diagram of a medical device implanted in a patient in which device the present invention can be implemented. As seen, this embodiment of the present invention is shown in the context of a pacemaker 2 implanted in a patient (not shown). The pacemaker 2 comprises a housing being hermetically sealed and biologically inert. Normally, the housing is conductive and may, thus, serve as an electrode. One or more pacemaker leads, where only two are shown in FIG. 1 namely a ventricular lead 6a and an atrial lead 6b, are electrically coupled to the pacemaker 2 in a conventional manner. The leads 6a, 6b extend into the heart 8 via a vein 10 of the patient. One or more conductive electrodes for receiving electrical cardiac signals and/or for delivering electrical pacing to the heart 8 are arranged near the distal ends of the leads 6a, 6b. As the skilled man in the art realizes, the leads 6a, 6b may be implanted with its distal end located in either the atrium or ventricle of the heart 8.

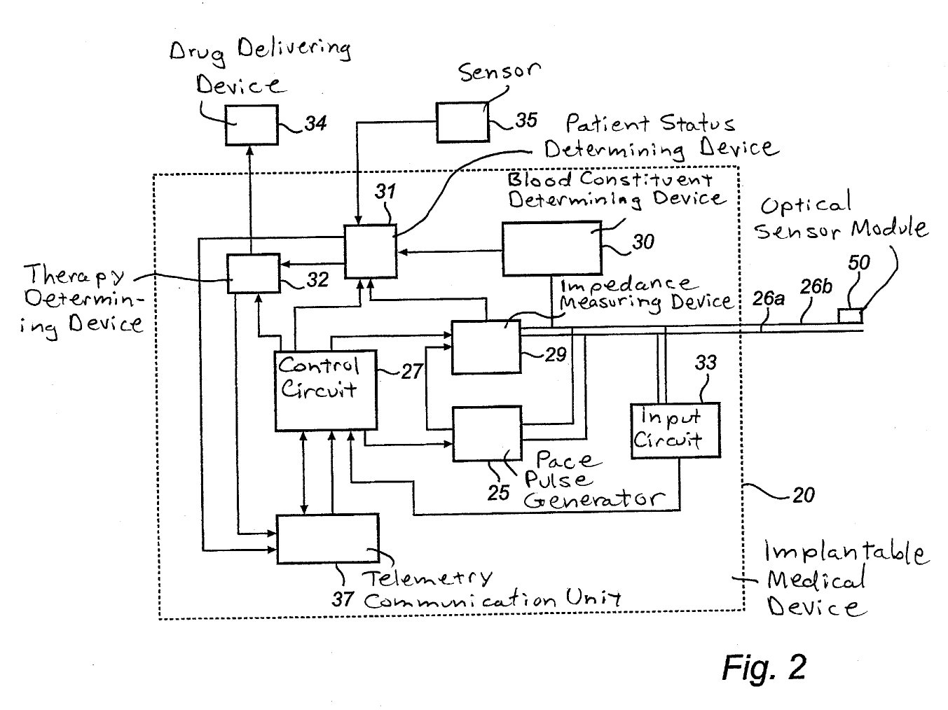

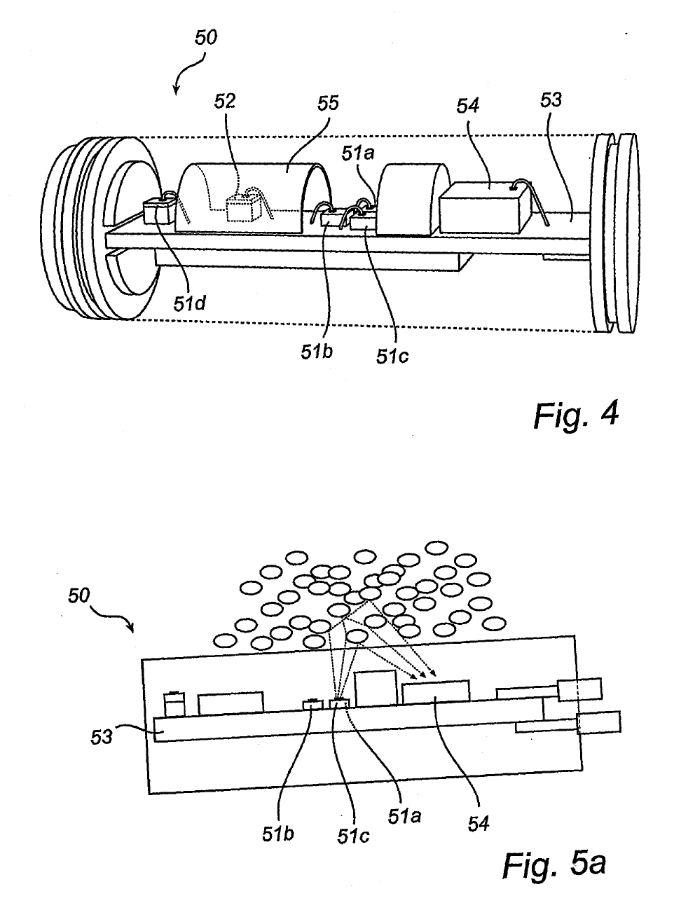

With reference now to FIG. 2, the configuration including the primary components of an embodiment of the present invention will be described. The illustrated embodiment comprises an implantable medical device 20, such as the pacemaker shown in FIG. 1, and leads 26a and 26b, of the same type as the leads 6a and 6b shown in FIG. 1, for delivering signals between the heart of the patient and the implantable medical device 20. The leads 26a, 26b may be unipolar or bipolar, and may include any of the passive or active fixation means known in the art for fixation of the lead to the cardiac tissue. As an example, the lead distal tip (not shown) may include a tined tip or a fixation helix. The leads 26a, 26b carry one or more electrodes (as described with reference to FIG. 1), such as a tip electrode or a ring electrode, arranged to, inter alia, transmit pacing pulses for causing depolarization of cardiac tissue adjacent to the electrode(-s) generated by a pace pulse generator 25 under influence of a control circuit 27 that includes (or is) a microprocessor. The control circuit 27 controls, inter alia, pace pulse parameters such as output voltage and pulse duration. An optical sensor module 50, which will be discussed in more detail with reference to FIG. 4, is further arranged in, for example, the atrial lead 26b adapted to measure and determine a hematocrit level of the blood and a SvO2 level of the blood.

Furthermore, the optical sensor module 50 is connected to a blood constituent determining device 30 adapted to obtain measured hematocrit values and SvO2 values from the optical sensor module 50, to determine a present hematocrit level by means of the at least one hematocrit value and to determine a present SvO2 level by means of the at least one SvO2 value. The hematocrit values may be obtained at a regular basis, i.e. at regular intervals, or continuously. Thereby, it is possible to obtain a sequence over time of hematocrit values and SvO2 values. Each value may be calculated as an average value over a predetermined number of values or of values obtained over a predetermined period of time or as a weighted average value over a predetermined number of values or of values obtained over a predetermined period of time.

A patient status determining device 31 is connected to the blood constituent determining device 30 and is adapted to determine a patient status based on an evaluation of the present hematocrit level and the present SvO2 level. The patient status may be used to derive a change of a condition of the patient. The patient status determining device 31 is also connected to sensors 35 of which only one is shown in FIG. 2 but, as the skilled person realizes, it may be more than one sensor. For example, a body temperature sensor, an activity level sensor (e.g. an accelerometer), a heart rate sensor, and/or a patient posture sensor. Thus, the patient status determination device 31 is capable of obtaining information on different physiological parameters such as body temperature, heart rate, patient posture, and activity level. Further, the patient determining device 31 is connected to an impedance measuring circuit 29 adapted to measure a trans-thoracic impedance using electrodes of the medical leads 26a and 26b and the housing of the implantable medical device. The impedance measuring device 29 is adapted to, during impedance measurement sessions, generate electrical signals to be applied between at least a first electrode and at least a second electrode and/or the housing and to measure the resulting impedance in the tissue between the at least first electrode and the at least second electrode to the applied electrical signals. The patient determining device 31 is adapted to obtain sensed impedance values continuously or at predetermined intervals, wherein at least one sequence over time of impedance values can be determined. The obtained sequence or sequences of the trans-thoracic impedance can be used to determine a minute ventilation in accordance with conventional manner known by the skilled person. Thereby, the evaluation of the obtained sensor values can be improved further by using the sequence or sequences of minute ventilation and the determination of a patient status to derive a change of a condition of the patient can be improved. The patient status may be determined by means of a reference value set including the hematocrit level and the SvO2 level. Predefined reference values can be stored in and obtained from an internal memory circuit, which may include a random access memory (RAM) and/or a non-volatile memory such as a read-only memory (ROM), of the control circuit 27. Alternatively, the predefined reference values may obtain from an external device via a telemetry communication unit 37. In another embodiment, the reference values may be created by the implantable medical device by performing reference measurement session during conditions found to be stable, for example, with respect to physiological parameters such as body temperature, heart rate, posture, activity, minute ventilation. The reference value set may constitute an indication of an initial patient status for use when determining a progression/regression of a patient condition, a disease or a trend of a certain parameter.

Moreover, a therapy determining device 32 is connected to the patient status determining device 31. The therapy determining device 32 may be adapted to obtain a patient status from the patient status determining device 31 to determine a therapy for the patient. Further, the therapy determining device 32 may be adapted to obtain a target range for the hematocrit level and the SvO2 level, respectively, and to compare the obtained target ranges with the present hematocrit level and the present SvO2 level, respectively, to determine a therapy for the patient including to determine a dosage of a drug based on the determined therapy. Accordingly, an optimal dosage for the patient may be determined taking into account changing outside factors such as the amount of exercise, food habits (consumption of coffee, salt, alcohol, etc.), which will change the amount of drugs required on a day to day basis Furthermore, according to this embodiment, the therapy determining device 32 is connected to a drug delivering device 34, which may be incorporated in the implantable medical device 20 or located outside the implantable medical device 20 and connected to the therapy determining device 32. The therapy determining device 32 is adapted to control the drug delivering device 34 so as to deliver a drug to the patient based on the determined dosage. In one embodiment, the drug delivering device 34 is a device for delivering diuretics, wherein the therapy determining device 32 is adapted to check whether a present hematocrit level is within the target range for the hematocrit level and to instruct the drug delivering device to adjust a delivery of diuretics such that the hematocrit level is maintained within the target range. In another embodiment, the drug delivering device 34 is a device for delivering a medication that affects the heart function of a patient. The therapy determining device 32 is adapted to monitor the SvO2 level by obtaining values from the patient status determining device 31 and the hematocrit level to check whether the SvO2 level is within a heart function target range for the SvO2 level, to check whether the present hematocrit level is within a heart function target range for the hematocrit level by obtaining values from the patient status determining device 31; to determine that a change in the SvO2 level is caused by a change of the heart function if the present hematocrit level is within the heart function target range; and to instruct the drug delivering device 34 to adjust a delivery of the medication such that the SvO2 level is maintained within the heart function target range if the SvO2 level change is determined to be caused by a changed heart function.

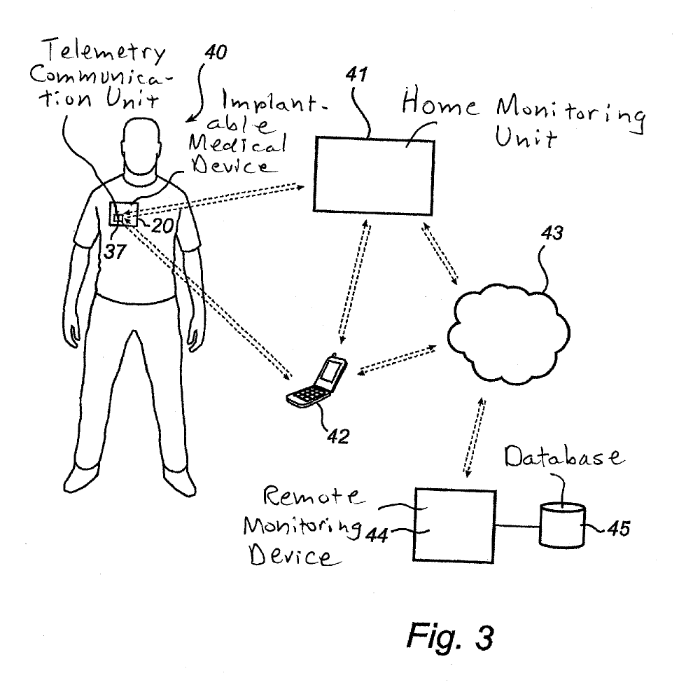

Detected signals from the patients heart are processed in an input circuit 33 and are forwarded to the microprocessor of the control circuit 27 for use in logic timing determination in known manner. The implantable medical device 20 is powered by a battery (not shown), which supplies electrical power to all electrical active components of the medical device 20. Data contained in, for example, the memory circuit of the control circuit 27, the patient status determining device 31, or the therapy determining device 32 can be transferred to a extracorporeal device such as a programmer (not shown) via a programmer interface (not shown) and the telemetry communication unit 37 for use in analyzing system conditions, patient information, etc. The telemetry communication circuit 37 is adapted for two-way communication with at least one extracorporeal device including a communication unit, see FIG. 3.

With reference now to FIG. 3, a system environment according to embodiments of the present invention will be discussed. An implantable medical device 20 as described above with reference to FIG. 2 is implanted in a patient 40. As discussed above, the implantable medical device may transfer data such as a determined dosage, a patient status or a change of a monitored condition or monitored physiological parameter to extracorporeal devices 41, 42, 44 via the RF communication unit 37. The extracorporeal devices 41, 42, 44, may communicate with each other via at least one external communication network such as wireless LAN (“Local Area Network”), GSM (“Global System for Mobile communications”), UMTS (“Universal Mobile Telecommunications System”). For a given communication method, a multitude of standard and/or proprietary communication protocols may be used. For example, and without limitation, wireless (e.g. radio frequency pulse coding, spread spectrum frequency hopping, time-hopping, etc.) and other communication protocols (e.g. SMTP, FTP, TCP/IP) may be used. Other proprietary methods and protocols may also be used. The communication unit 37 is adapted for two-way communication with an extracorporeal home monitoring unit 41, which may be located in the patients home, including a display means such as a display screen and input means such as a mouse and a keyboard and/or a user equipment 42 such as a mobile phone, a personal digital assistant, or a pager. Further, the user equipment 42 may be adapted to be carried by the patient similar to wrist watch or to be attached at a belt. The communication unit 37 may also communicate with a remote monitoring device 44, e.g. a PC, located at, for example, a care institution via the home monitoring unit 41 and/or via the user equipment 42 via a communication network as described above or via Internet. The monitoring device 44 may be connected to a database 45 for storage of patient data.

In embodiments of the present invention, the patient status determining device 31 may transfer patient status data and/or trend data of the different measured parameters including hematocrit, SvO2, body temperature, heart rate, activity level, patient posture and/or minute ventilation to the extracorporeal devices 41, 42, 44 via the telemetry communication unit 37. As the skilled person realizes, there are other physiological/hemodynamical parameters that may be monitored such as cardiovascular pressure, cardiac output, or PR interval (or AR interval). The patient is hence able to monitor a progression/regression of a disease and/or a trend of a certain parameter or certain parameters at the user equipment 42 and/or the home monitoring unit 41. This information may also be transferred to the monitoring device 44 at the care institution via the communication network 43, either directly or via the home monitoring unit 41 or the user equipment 42, thereby allowing a physician to view a progression/regression of a disease and/or a trend of a certain parameter or certain parameters. The trend may either be displayed to the physician at a follow-up of the patient or upon an inquiry sent to the implantable medical device 20 from the monitoring device 44 via the communication network 43 and the home monitoring unit 41. The information can be used to guide long term therapy, such as if the patient should be equipped with a different device or if the type of medication should be changed. The information may also be used by the physician to determine a dosage of a drug.

Furthermore, predetermined upper or lower limits may be set for one of, some of, or all of the parameters including hematocrit, SvO2, body temperature, heart rate, activity level, patient posture and/or minute ventilation within which limits they are allowed to fluctuate between. Thus, different combinations of parameters can be used to provide indications of different conditions and a set of criteria may be defined for that purpose. Each criterion may give rise to an alert signal and there may hence be a number of different signals each signalling the crossing of a limit for a certain parameter. The patient status determining device 31 may send such an alert signal to the user equipment 42 and/or the home monitoring unit 41 informing the patient that he or she should see his/her physician. In another embodiment, the medical device 20 may include an alarm means adapted to cause the device to vibrate or to deliver a beeping sound in order to alert the patient of the situation, the alarm means may be integrated into the control circuit 27 or the patient status determining device 31. Alternatively, or as a complement, this information together with the progression of the trend may be sent with an alert signal to the physician to be viewed on the monitoring device 44 so that he or she can decide whether the patient should be called in for a visit. For example, hematocrit is a good parameter for establishing how well the kidney is functioning and may thus be used as an indicator of the kidney function as well as for patients with kidney disease to guide their medication. Furthermore, many heart failure patients has co-morbidities and the same symptom can originate for different errors in the patient, dyspnea for instance can be a sign of volume overload in the lung but can also be a sign of poor oxygenation of the patient due to low hematocrit. A trend over several physiological parameters including hematocrit, SvO2, body temperature, heart rate, activity level, patient posture and/or minute ventilation will provide the physician with a tool to establish the cause of a deterioration and grade of the severity of the problem. If the physician receives an alert signal together with collected data of the parameters, the physician/nurse may rate the level of acuteness of the deterioration and be guided whether the patient have to visit the hospital or care institution at once or within the next few weeks, or only be prescribed a new medication. It may also be established in an early phase which physician branch (nephrologist, cardiologist, pulmonologist, internal medicine) the patient should see if a hospital visit is required. It would also be beneficial in an in-clinic scenario where the information could eliminate certain pathophysiological factors and thus eliminate tests thereby reducing costs and provide the physician with the relevant information. Also, in a regular follow-up it would provide insight to patients general health and help guide the physician of the overall therapy. In a remote follow-up scenario it would provide the physician with physiological/hemodynamic information to provide between quality of the follow-up. For example, if the SvO2 level decreases but the hematocrit level remains more or less the same, it is an indication that there is something wrong with the absorption of oxygen. If SvO2 remains more or less the same but the level of hematocrit goes down and the heart rate increases, there is indication for anaemia. Further, if SvO2 remains within normal limits, hematocrit remains within normal limits, body temperature increases and heart rate increases, there is an indication of that the patient has an infection.

According to other embodiments, the therapy determining device 32 may transfer data including a determined dosage to the extracorporeal devices 41, 42, 44 via the telemetry communication unit 37. The patient is able to view a determined dosage by means of the user equipment 42 or the monitoring device 41 and may thus be informed of, for example, a change of dosage. This is of great use since many outside factors such as the amount of exercise, food habits (consumption of coffee, salt, alcohol, etc.) will change the amount of drugs required on a day to day basis. Thus, the patient will obtain dosage information such that he or she will be able to adjust the dosage in order to cope with the above mentioned changing outside factors. The patient is thereby able to avoid over consumption as well as under consumption. An over consumption is unbeneficial since the drug often is associated with bi-effects and an under consumption will lead to an ineffective therapy. The dosage information may also, or instead, be transferred to the monitoring device 44 at the care institution thereby allowing a physician to monitor the medication of the patient.

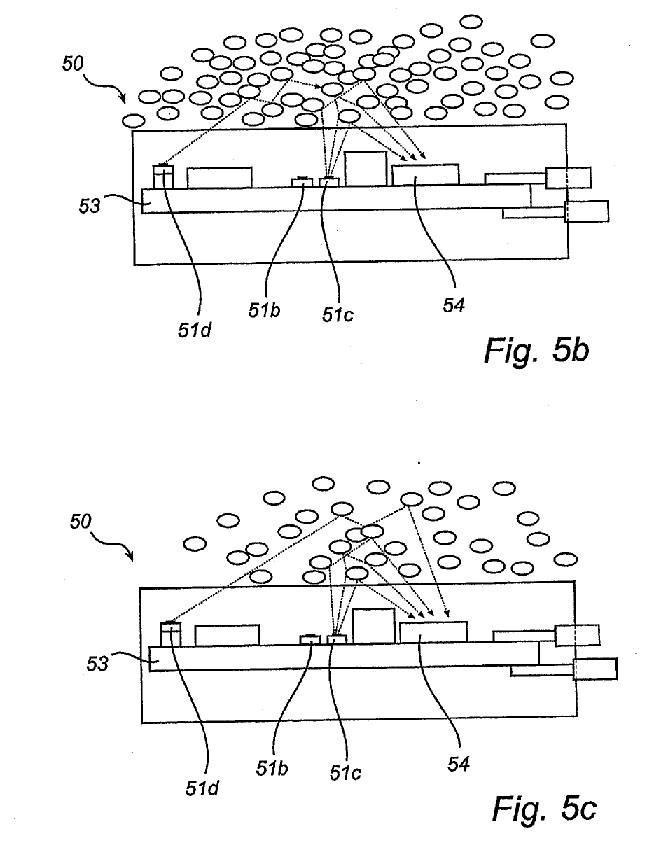

Referring now to FIG. 4, the optical sensor module 50 will be described. The sensor module is based on the different light reflecting properties of oxygenated and reduced hemoglobin. The measurements are influenced by, inter alia, blood flow and erythrocyte shape. The use of two or more wavelength may compensate for these effects. The optical sensor module 50 is integrated in a medical lead, for example, the atrial lead and is hermetically sealed inside a tube, for example, of sapphire. According to an embodiment, four LEDs 51a, 51b, 51c, 51d at wavelengths 670, 700, 805, and 805 nm, respectively, and a built in calibration photodiode 52 are arranged on a substrate 53 in the module 50. Further, a photodiode 54 is adapted to receive the light emitted from the LEDs 51a, 51b, 51c, 51d and reflected by the blood cells. According to this embodiment, the first, second, third LED 51a, 51b, and 51c are adapted to emit light at wavelengths 670, 700, and 805 nm to measure oxygen saturation (SvO2), see FIG. 5a. The LEDs 51c and 51d are adapted to emit light at wavelength 805 nm to measure hematocrit, see FIG. 5b in which it is schematically illustrated the light paths at a higher degree of hematocrit and FIG. 5c in which it is schematically illustrated the light paths at a lower degree of hematocrit. In FIG. 5d, the calibration is schematically shown. As can be seen, the LEDs 51a, 51b, 51c, and 51d emit light against the reflective surface 55, which reflects the light against the calibration photodiode 52. The theoretical background of the optical sensor and of the different light reflecting properties of oxygenated and reduced hemoglobin as well as the influence of, inter alia, blood flow and erythrocyte shape on the measurements are described in detail in U.S. Pat. No. 4,114,604, Shaw R. F. et al., and are therefore not repeated here in further detail.

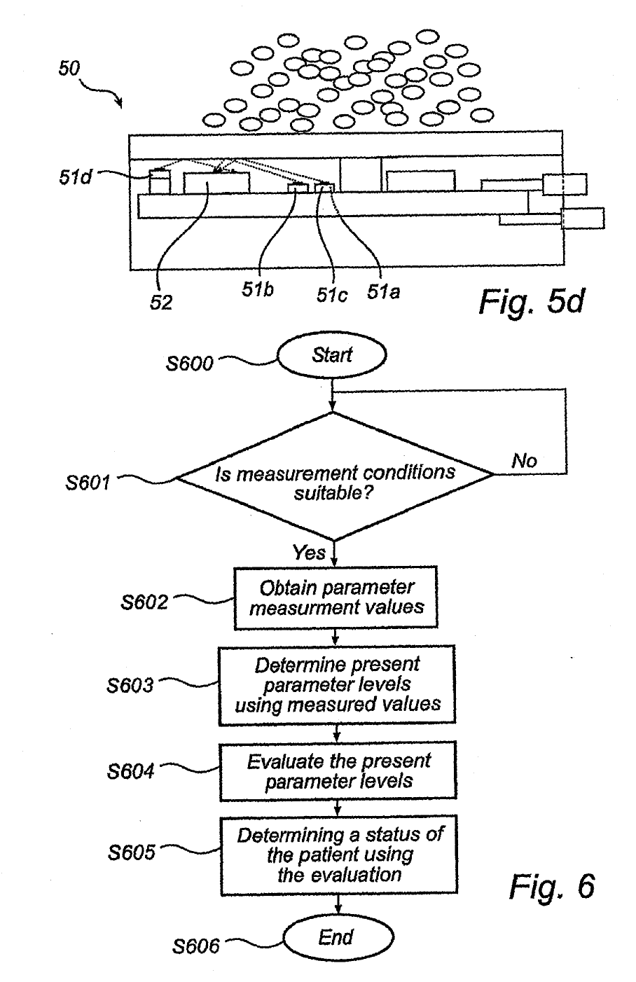

Referring now to FIG. 6, a high-level description of the method according to the present invention will be given. The patient status determining device 31 may optionally perform a check whether the measurement conditions during which the measurements are performed are suitable, i.e. whether the conditions are such that reliable and reproducible signals can be obtained. For example, a condition for considering the measured parameter values as usable in the determination of, for example, a patient status and/or a dosage of medication may be that a sensed activity level of the patient is within a predetermined range, that the patient is within a certain predetermined posture, or that the body temperature is within a predetermined range. The parameters can be sensed by means of sensors incorporated in the medical device in accordance with conventional practice within the art. In an alternative embodiment, the measurements are initiated when the measurements conditions are approved, that is, the measurement session is initiated only if, for example, the activity level signal is within the predetermined range. In case of this measurement condition check, a measurement condition obtaining procedure step is executed before the actual check is performed. Thus, optionally, a measurement condition obtaining procedure step and measurement condition check S601 may be performed after the procedure to derive a condition of a patient is started at step S600. The procedure may be executed regularly, continuously, at a request from the patient received via the user equipment 42 or the home monitoring unit 41, or at a request from a physician via the monitoring device 44. If the measurement conditions are found to be suitable, measurement values of physiological parameters are obtained, at regular intervals or continuously, at step S602. As mentioned above, there are a number of different physiological parameters that can be measured for use in determining, for example, a patient status or a dosage including hematocrit, SvO2, body temperature, heart rate, patient posture, and/or minute volume (using e.g. transthoracic impedance). In a preferred embodiment, the hematocrit and SvO2 are measured at regular intervals or continuously.

Optionally, a validity check may be performed in order to check or judge whether the obtained parameter values are reasonable or valid. This can be performed, for example, by checking that the obtained value is within a preset range including the preceding value. If the obtained value is found to be invalid, i.e. the value is outside the preset range, the value or signal is rejected. In one embodiment, a new measurement session is initiated after a delay period of a predetermined length and if this is repeated a preset number of times without a valid signal has been obtained, the procedure returns to the idle mode.

Then, at step S603, present parameter levels are determined. The levels may be determined as a mean value of respective measured parameter value over a predetermined period of time or as mean of a predetermined number of values. Further, the parameter levels may be relative or absolute. The determined parameter levels may be stored as a trend over the progression of the parameter. Subsequently, at step S604, an evaluation of the determined parameters are performed. For example, reference values for the evaluated parameters, e.g. hematocrit and SvO2, may be obtained by the patient status determining device 31 from an internal memory of, for example, the control circuit 27 or the patient status determining device 31, or from the database 45 via the communication network 43. The reference values may constitute an initial patient status and may thus be used for comparison with later levels of the parameters to evaluate the trend. For example, it may be determined that the level SvO2 decreases and that the hematocrit level is more or less the same, or that SvO2 remains more or less the same but the level of hematocrit decreases and the heart rate increases, or that SvO2 is within normal limits, hematocrit is within normal limits, body temperature increases and heart rate increases. This data is used to determine a patient status in step S605. For example, if the level SvO2 is determined to decrease but the hematocrit level remains more or less the same, it is an indication the there is something wrong with the absorption of oxygen. Further, it is determined that SvO2 remains more or less the same but the level of hematocrit goes down and the heart rate increases, there is indication for anaemia. If SvO2 is determined to be within normal limits, hematocrit to be within normal limits, body temperature to increase and heart rate to increase, there is an indication of that the patient has an infection. The procedure is then terminated at step S606.

As the skilled man realizes, the steps described above with reference to FIG. 6 are not necessarily executed in the given order, certain steps may be executed simultaneously or in reversed order.

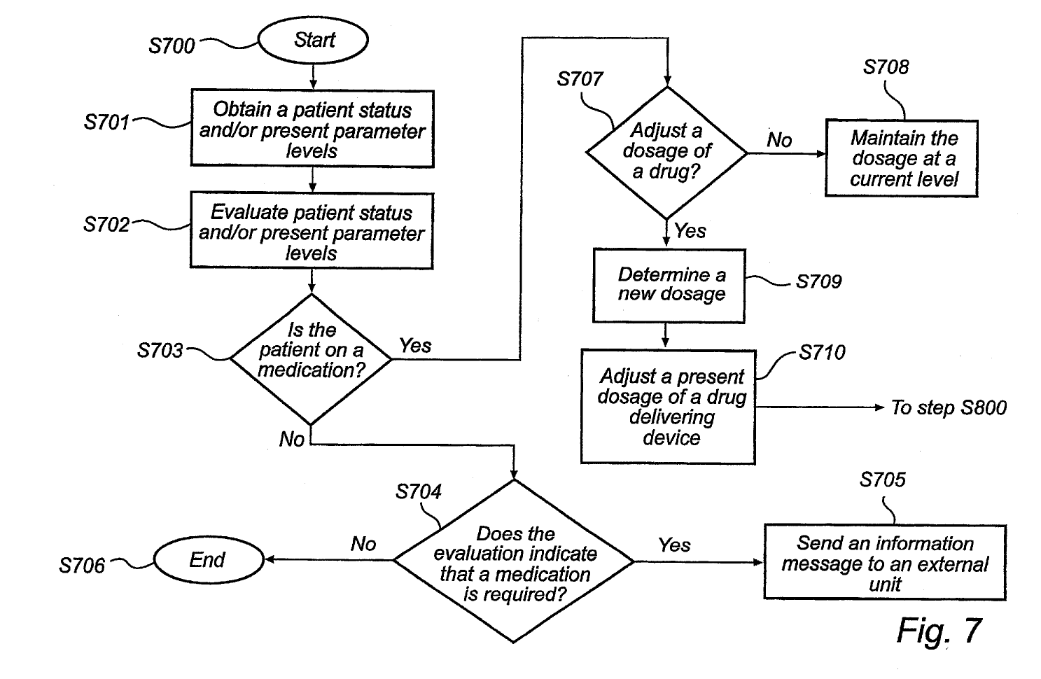

With reference now to FIG. 7, a high-level description of an exemplary embodiment of the method according to the present invention will be given. First, at step S700, the procedure is initiated. The procedure can be initiated and performed by the therapy determining device 32, for example, at regular intervals or by receipt of an instruction from the control circuit 27. At step S701, the therapy determining device 32 obtains a patient status and/or present parameter levels from the patient status determining device 31. Then, at step S702, the patient status and/or the present parameter values are evaluated. Reference data may be obtained for use in this evaluation from, for example, a patient medication protocol stored in an internal memory of the therapy determining device 32 or from a patient register in the database 45 via the communication unit 37. For example, a target range for the hematocrit level and the SvO2 level, respectively, can be obtained and these target ranges can be compared with the present hematocrit level and the present SvO2 level, respectively, to determine a therapy for the patient. That is, whether a present value exceeds an upper limit of the target range or falls below a lower limit of the target range for the hematocrit and/or the SvO2. Thereafter, at step S703, it is checked whether the patient is on a medication. This information may, for example, be included in the patient medication protocol stored in the internal memory of the therapy determining device 32 or in the patient register in the database 45. If no, the procedure proceeds to step S704 where it is checked whether the evaluation indicates that a therapy/medication is required. For example, if it is verified that a present SvO2 level is not within a heart function target range for the SvO2 level and that the present hematocrit level is within a heart function target range for the hematocrit level, it may be determined that the change in the SvO2 level is caused by a change of the heart function and thus that a medication affecting the heart function of the patient may be required. Then, at step S705, this information is sent to an external device 41, 42, or 44. The information can be sent as a alert signal to the patient to the user equipment 42 or the home monitoring unit 41 informing the patient of the situation and/or to the monitoring device 44 of the care institution informing a physician that the patient should be called in for a medical examination. On the other hand, if it is determined that no/therapy is required medication, the procedure proceeds to step S706, where it is terminated.

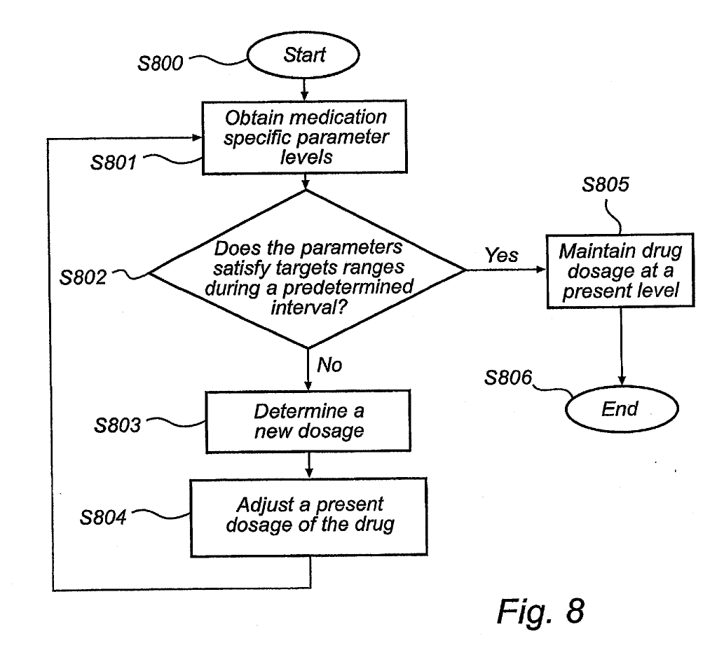

If the procedure in step S703 finds that the patient is on a medication, for example, that the patient is provided with a heart function affecting drug, it proceeds to step S707 where a check whether the dosage should be adjusted is performed on basis of the evaluation. For example, if it is verified that a present SvO2 level is within a heart function target range for the SvO2 level and that the present hematocrit level is within a heart function target range for the hematocrit level, it may be determined that the change in the SvO2 level is within normal variations and that no change of medication is required. In his case, the procedure proceeds to step S708 where the a present drug dosage is maintained. However, if it, on the other hand, is verified that a present SvO2 level is not within a heart function target range for the SvO2 level and that the present hematocrit level is within a heart function target range for the hematocrit level, it may be determined that the change in the SvO2 level is caused by a change of the heart function and thus that change of the heart function affecting drug dosage is required. In such a case, the procedure proceeds to step S709 where a new dosage of a drug is determined. Information regarding a present drug dosage is held in the patient medication protocol and if a new drug dosage is determined, the protocol may be updated with this new information. This new dosage information may be communicated to the patient by means of the user equipment 42 or the home monitoring unit 41 and/or to the physician by means of the monitoring device 44. In this described embodiment, the therapy determining device 32 is connected to a drug delivering device 34, which may be implanted, and, in step S710, the present dosage is adjusted in the drug delivering device 34. Subsequently, the procedure proceeds to an iterative drug delivery adjustment procedure, which will be described with reference to FIG. 8 hereinafter.

As the skilled man realizes, the steps described above with reference to FIG. 7 are not necessarily executed in the given order, certain steps may be executed simultaneously or in reversed order and certain steps may be left out. For example, step S703 may be left out since the procedure may have received this information as an update when the patient initiates his or hers medication.

Turning now to FIG. 8, the drug delivery adjustment procedure according to an embodiment of the present invention will be described. This procedure may be delayed a predetermined period of time to allow the new adjusted dosage of the drug to give the desired effect. At step S800, the procedure is initiated. Then, at step S801, the therapy determining device 32 obtains parameters specific for the particular therapy or drug for which the dosage was changed. For example, in the above given example with a drug affecting the heart function of the patient, the hematocrit and the SvO2 levels are monitored and obtained regularly or continuously. Thereafter, at step S802, it is checked whether the obtained parameters satisfy the target ranges for the respective parameters defined in the medication protocol, i.e. whether the monitored parameters are within the target ranges, respectively, during a predetermined period of time or for a number of consecutive measurements. If no, it is determined that a new dosage of the drug must be calculated. For example, if it is verified that a present SvO2 level is not within a heart function target range for the SvO2 level and that the present hematocrit level is within a heart function target range for the hematocrit level despite the preceding dosage adjustment, it may be determined that the change in the SvO2 level is caused by a change of the heart function and thus that a further change of the heart function affecting drug dosage is required. In such a case, the procedure proceeds to step S803 where a new dosage of a drug is determined. Information regarding a present drug dosage is held in the patient medication protocol and when a new drug dosage is determined, the protocol may be updated with this new information. This new dosage information may be communicated to the patient by means of the user equipment 42 or the home monitoring unit 41 and/or to the physician by means of the monitoring device 44. In this described embodiment, the therapy determining device 32 is connected to a drug delivering device 34, which may be implanted, and, in step S709, the present dosage is adjusted in the drug delivering device 34. However, this information and/or updating procedure may be performed after the iteration procedure has been finished. Then, at step S804, the present dosage is adjusted in the drug delivering device 34. The procedure then returns to step S801. On the other hand, if it is found that the monitored parameters are within target ranges, for example, if it is verified that a present SvO2 level is within a heart function target range for the SvO2 level and that the present hematocrit level is within a heart function target range for the hematocrit level, it may be determined that the change in the SvO2 level is within normal variations and that no further change of medication is required, the procedure proceeds to step S805 where the dosage is maintained at the present level. Finally, the procedure is terminated at step S806.

As those skilled in the art will realize, the steps described above with reference to FIG. 8 are not necessarily executed in the given order or certain steps may be executed simultaneously.

Although modifications and changes may be suggested by those skilled in the art, it is the intention of the inventors to embody within the patent warranted heron all changes and modifications as reasonably and properly come within the scope of their contribution to the art.

1. An implantable medical device for monitoring a hematocrit level and a SvO2 level of a patient comprising:
at least one medical lead including an optical sensor module that measures at least one hematocrit value and at least one SvO2 value using at least first, second, and third light radiation wavelengths;
a blood constituent determining device that obtains measured hematocrit values and SvO2 values, and that determines a present hematocrit level from said at least one hematocrit value and that determines a present SvO2 level from said at least one SvO2 value; and
a patient status determining device that determines a patient status by evaluating said present hematocrit level and said present SvO2 level, to derive a change of a condition of said patient, therefrom, and that makes said patient status or said change available as an output.

2. The implantable medical device according to claim 1, wherein said blood constituents determining device obtains measured hematocrit values and SvO2 values continuously or at predetermined intervals, from which at least one sequence over time of hematocrit levels and SvO2 levels, respectively, are determined.

3. The implantable medical device according to claim 1, further comprising a therapy determining device that obtains a target range for said hematocrit level and said SvO2 level, respectively, and that compares said obtained target ranges with said present hematocrit level and said present SvO2 level, respectively, to determine a therapy for said patient.

4. The implantable medical device according to claim 3, further comprising a temperature sensor that senses a body temperature of said patient, and wherein said therapy determining device determines said therapy also based on said body temperature.

5. The implantable medical device according to claim 4, wherein said patient status determining device is configured:
obtain sensed body temperature values continuously or at predetermined intervals, wherein at least one sequence over time of body temperature values can be determined;
use said sensed body temperature values in said evaluation; and
determine said patient status based on said evaluation, wherein a change of a condition of said patient can be derived.

6. The implantable medical device according to claim 3, further comprising an activity sensor that senses an activity level of said patient.

7. The implantable medical device according to claim 6, wherein said patient status determining device is configured to:
obtain sensed activity levels continuously or at predetermined intervals, wherein at least one sequence over time of an activity level can be determined;
use said sensed activity levels in said evaluation; and
determine said patient status based on said evaluation, wherein a change of a condition of said patient can be derived.

8. The implantable medical device according to claim 3, further comprising an impedance measuring circuit that measures a transthoracic impedance, said impedance measuring circuit connected to electrodes of said at least one medical lead and/or to a housing of said implantable medical device, said impedance measuring circuit to, during impedance measurement sessions, generating electrical signals applied between at least a first electrode and at least a second electrode; and measuring the impedance in the tissue between said at least first electrode and said at least second electrode to the applied electrical signals, and wherein said therapy determining device determines said therapy also based on said transthoracic impedance.

9. The implantable medical device according to claim 8, wherein said patient status determining device is configured to:
obtain sensed impedance values continuously or at predetermined intervals, wherein at least one sequence over time of impedance values can be determined;
determine at least one sequence of a minute ventilation of said patient using said impedance values;
use said sequence of minute ventilation in said evaluation; and
determine said patient status based on said evaluation, wherein a change of a condition of said patient can be derived.

10. The implantable medical device according to claim 3, further comprising a heart rate sensor that senses a heart rate of said patient, and wherein said therapy determining device determines said therapy also based on said heart rate.

11. The implantable medical device according to claim 10, wherein said patient status determining device is adapted, and wherein said therapy determining device determines said therapy also based on said heart rate:
obtain sensed heart rate level values continuously or at predetermined intervals, wherein a sequence over time of heart rate levels values can be determined;
include said sensed heart rate values in said evaluation; and
determine said patient status based on said evaluation, wherein a change of a condition of said patient can be derived.

12. (canceled)

13. The implantable medical device according to claim 3, wherein said therapy determining device determines a dosage of a drug based on said determined therapy.

14. The implantable medical device according to claim 13, comprising a drug delivering device connected to said therapy determining device, and wherein said therapy determining controls said drug delivering device so as to deliver a drug to said patient based on said determined dosage.

15. The implantable medical device according to claim 3, further comprising a telemetry communication unit configured for two-way communication with at least one extracorporeal device including a communication unit, said telemetry communication unit being connected to said patient status determining device and said therapy determining device, and wherein patient status related data and/or therapy related data are transferred from said communication device to said extracorporeal device or vice versa.

16. The implantable medical device according to claim 15, wherein said therapy determining device transfers data including said determined dosage to said extracorporeal device via said telemetry communication unit, such that said patient is informed of said determined dosage.

17. The implantable medical device according to claim 15, wherein said extracorporeal device is a monitoring unit or user equipment.

18. The implantable medical device according to claim 15, wherein said patient status related data and/or said therapy related data is transferred to said extracorporeal device at regular intervals.

19. The implantable medical device according to claim 15, wherein said extracorporeal unit is connected to a communication network, wherein said patient status related data and/or said therapy related data are transferred via said network to a monitoring device located at a care institution connected to said network such that a physician is informed of a patient status and/or a determined therapy or dosage.

20. The implantable medical device according to claim 1, wherein said patient status determining device is configured to:
monitor at least one of the hematocrit level, the SvO2 level, body temperature; heart rate; activity level, and/or the minute ventilation; and
determine whether said at least one monitored parameter is within predetermined lower and upper limits for respective parameter.

21. The implantable medical device according to claim 20, wherein said patient status determining device provides said patient with a notification that a monitored parameter has exceeded or fallen below said predetermined limits, respectively, by sending an alert signal.

22. The implantable medical device according to claim 21, wherein said alert signal is sent to said extracorporeal device or wherein said alert signal is sent to a vibration unit of said implantable medical device (2; 20) causing it to vibrate.

23. The implantable medical device according to claim 21, wherein said patient status determining device transfers said alert signal and/or information related to a development of said monitored parameter over time to an extracorporeal device via a telemetry communication unit, such that said patient is informed of said event.

24. The implantable medical device according to claim 20, wherein said patient status determining device transfers said alert signal and/or information related to a development of said monitored parameters over time to a monitoring device via a network, such that a physician is informed of said event.

25. The implantable medical device according to claim 3, wherein said therapy determining device calibrates a level of medication by controlling a drug delivering device so as to adjust a delivery of a drug to said patient based on said comparison between said obtained target ranges with said present hematocrit level and said present SvO2 level, respectively.

26. The implantable medical device according to claim 25, wherein said therapy determining device calibrates a level of medication by controlling said drug delivering device so as to adjust a delivery of a drug to said patient based on said measured parameters including any combination of: the body temperature; the heart rate; the activity level, or the minute ventilation.

27. The implantable medical device according to claim 3, wherein said drug delivering device is a device for delivering diuretics, and wherein said therapy determining device is configured to check whether said present hematocrit level is within said target range for the hematocrit level; and to instruct said drug delivering device to adjust a delivery of diuretics such that said hematocrit level is maintained within said target range.

28. The implantable medical device according to claim 3, wherein said drug delivering device is a device for delivering a medication that affects the heart function of a patient, and wherein said therapy determining device is configured to:
monitor said SvO2 level and said hematocrit level, to check whether said SvO2 level is within a heart function target range for the SvO2 level;
check whether said present hematocrit level is within a heart function target range for the hematocrit level;
determine that a change in said SvO2 level is caused by a change of the heart function if said present hematocrit level is within said heart function target range; and
instruct said drug delivering device to adjust a delivery of said medication such that said SvO2 level is maintained within said heart function target range if said SvO2 level change is determined to be caused by a changed heart function.

29. The implantable medical device according to claim 1, wherein said patient status determining device is adapted to:
obtain at least one reference value for: hematocrit, SvO2, body temperature; heart rate; the activity level, and/or minute ventilation; and
use said at least one reference value in said evaluation.

30. The implantable medical device according to claim 29, wherein said blood constituent determining device is configured to:
determine a reference hematocrit level, a reference SvO2 level; a reference body temperature; a reference heart rate; a reference activity level, and/or a reference minute ventilation using at least one respective measured parameter value; and
store said determined respective reference values in a storage unit.

31. A method for monitoring a hematocrit level and a SvO2 level of a patient in an implantable medical device connectable to at least one medical lead including an optical sensor module adapted to measure at least one hematocrit value and at least one SvO2 value by means of at least first, second, and third light radiation wavelengths, comprising the steps of:
measuring hematocrit values and SvO2 values;
determining a present hematocrit level by means of said at least one hematocrit value and to determine a present SvO2 level by means of said at least one SvO2 value; and
determining a patient status based on an evaluating said present hematocrit level and said present SvO2 level, including deriving a change of said patient status.

32. The method according to claim 31, wherein said step of measuring hematocrit values and SvO2 values comprises the step of measuring hematocrit values and SvO2 values at predetermined intervals, wherein at least one sequence over time of hematocrit levels and SvO2 levels, respectively, is determined.

33. The method according to claim 31, further comprising the steps of:
obtaining a target range for said hematocrit level and said SvO2 level, respectively;
comparing said obtained target ranges with said present hematocrit level and said present SvO2 level, respectively; and
determining a therapy for said patient based on said comparison.

34. The method according to claim 31, further comprising the step of sensing a body temperature of said patient.

35. The method according to claim 34, wherein said step of determining a patient status comprises the steps of:
obtaining sensed body temperature values continuously or at predetermined intervals, wherein at least one sequence of body temperature values over time can be determined;
using said sensed body temperature values in said evaluation;
determining said patient status based on said evaluation, deriving a change of a condition of said patient.

36. The method according to any one of preceding claims 31, further comprising the step of sensing an activity level of said patient.

37. The method according to claim 36, wherein said step of determining a patient status comprises the steps of:
obtaining sensed activity level values continuously or at predetermined intervals, wherein at least one sequence of body temperature values over time can be determined;
using said sensed activity level values in said evaluation;
determining said patient status based on said evaluation, wherein a change of a condition of said patient can be derived.

38. The method according to claim 31, further comprising the steps of measuring a trans-thoracic impedance, including, during impedance measurement sessions,
generating electrical signals to be applied between at least a first electrode and at least a second electrode of electrodes of said at least one medical lead and/or said housing of said implantable medical device; and
measuring the impedance in the tissue between said at least first electrode and said at least second electrode to the applied electrical signals.

39. The method according to claim 38, wherein said step of determining a patient status comprises the steps of:
obtaining sensed impedance values continuously or at predetermined intervals, wherein at least one sequence of impedance values overtime can be determined;
determining at least one sequence of a minute ventilation of said patient using said impedance values;
using said determined sequence of minute ventilation in said evaluation;
determining said patient status based on said evaluation, and deriving a change of a condition of said patient.

40. The method according to claim 31, further comprising the step of sensing a heart rate of said patient.

41. The method according to claim 40, wherein said step of determining a patient status comprises the steps of:
obtaining sensed heart rate values continuously or at predetermined intervals, wherein at least one sequence of heart rate values over time can be determined;
using said sensed heart rate values in said evaluation;
determining said patient status based on said evaluation, and deriving a change of a condition of said patient.

42. The method according to claim 33, wherein said step of determining a therapy comprises using at least one of, or all of the following parameters: body temperature, heart rate, activity level, and minute ventilation.

43. The method according to claim 33, wherein said step of determining a therapy comprises determining a dosage of a drug based on said determined therapy.

44. The method according to claim 43, comprising connecting said implantable medical device to a drug delivering device said method further comprising controlling said drug delivering device to deliver a drug to said patient based on said determined dosage.

45. The method according to claim 31, wherein said implantable medical comprises a telemetry communication unit adapted for two-way communication with at least one extracorporeal device, said telemetry communication unit being connected to said patient status determining device and said therapy determining device, wherein said method further comprises transferring patient status related data and/or therapy related data from said communication device to said extracorporeal device or vice versa.

46. The method according to claim 45, further comprising transferring data including said determined dosage to said extracorporeal device via said telemetry communication unit, wherein said patient can be informed of said determined dosage.

47. The method according to claim 45 comprising employing a monitoring unit or a user equipment as said extracorporeal device.

48. The method according to claim 45, further comprising transferring said patient status related data and/or said therapy related data to said extracorporeal device at regular intervals.

49. The method according to claim 45, comprising connecting said extracorporeal unit to a communication network, and wherein said method further comprises said patient status related data and/or said therapy related data via said network to a monitoring device connected to said network, to inform a physician of a patient status and/or a determined therapy or dosage.

50. The method according to claim 31, wherein said step of determining a patient status further comprises the steps of:
monitoring at least one of the following parameters: hematocrit level, SvO2 level, body temperature, heart rate, the activity level, and minute ventilation; and
determining whether said monitored parameter is within predetermined lower and upper limits.

51. The method according to claim 50, wherein said step of determining a patient status further comprises the step of:
providing said patient with a notification that a monitored parameter has exceeded or fallen below said predetermined limits, respectively, by sending an alert signal.

52. The method according to claim 50, further comprising sending said alert signal to said extracorporeal device or to a vibration unit of said implantable medical device causing it to vibrate.

53. The method according to claim 50, further comprising transferring said alert signal and/or information related to a development of said hematocrit level and said SvO2 level over time to said extracorporeal device via said telemetry communication unit, to inform said patient of said event.

54. The method according to claim 33, further comprising transferring said alert signal and/or information related to a development of said hematocrit level and said SvO2 level over time to said monitoring device via a network, to inform a physician of said event.

55. The method according to claim 33, further comprising the step of calibrating a level of medication by controlling said drug delivering device to adjust a delivery of a drug to said patient based on said comparison between said obtained target ranges with said present hematocrit level and said present SvO2 level, respectively.

56. The method according to claim 55, wherein said step of calibrating further comprises:
calibrating a level of medication by controlling said drug delivering device to adjust a delivery of a drug to said patient based on said measured parameters including any combination of: the body temperature, the heart rate, the activity level, or the minute ventilation.

57. The method according to claim 33, wherein said drug delivering device is a device for delivering diuretics, and wherein said method further comprises the steps of:
checking whether said present hematocrit level is within said target range for the hematocrit level; and
instructing said drug delivering device to adjust a delivery of diuretics such that said hematocrit level is maintained within said target range.

58. The method according to claim 33, wherein said drug delivering device is a device for delivering a medication that affects the heart function of a patient, said method further comprising the steps of:
monitoring said SvO2 level and said hematocrit level;
checking whether said SvO2 level is within a heart function target range for the SvO2 level;
checking whether said present hematocrit level is within a heart function target range for the hematocrit level;
determining that a change in said SvO2 level is caused by a change of the heart function if said present hematocrit level is within said heart function target range; and
instructing said drug delivering device to adjust a delivery of said medication such that said SvO2 level is maintained within said heart function target range if said SvO2 level change is caused by a changed heart function.

59. The method according to claim 31, wherein said step of evaluating further comprises the steps of:
obtaining at least one reference value for the hematocrit, the SvO2, the body temperature, the heart rate, the activity level, and/or the minute ventilation; and
using said at least one reference value in said evaluation.

60. The method according to claim 59, further comprising the steps of:
determining a reference hematocrit level, a reference SvO2 level, a reference body temperature, a reference heart rate, a reference activity level, and/or a reference minute ventilation; and
storing said determined respective reference values.

61. (canceled)

62. A computer-readable medium encoded with programming instructions loadable into an internal memory of an implantable medical device that is connected to at least one medical lead that carries an optical sensor module that measures at least one hematocrit value and at least one SvO2 value using first, second and third light radiation wavelengths, said programming instructions causing said implantable medical device to monitor a hematocrit level and an SvO2 level, by causing said implantable medical device to:
measure hematocrit values and SvO2 values;
determine a present hematocrit value from at least one of said hematocrit values and to determine a present SvO2 value from at least one of said SvO2 values; and
determine a patient status by evaluating said present hematocrit value and said present SvO2 value, including derivation of a change of said patient status.
